(19) United States
(12) Patent Application Publication
(10) Pub. No.: US 2016/0363580 A1
(43) Pub. Date: Dec. 15, 2016

(54) Methods of Metabolic Kinetic Phenotyping and Uses Thereof

(71) Applicants: Nicolaas E. Deutz, College Station, TX (US); Marielle P. Engelen, College Station, TX (US)

(72) Inventors: Nicolaas E. Deutz, College Station, TX (US); Marielle P. Engelen, College Station, TX (US)

(73) Assignee: The Texas A&M University System, College Station, TX (US)

(21) Appl. No.: 15/180,571

(22) Filed: Jun. 13, 2016

Related U.S. Application Data
(60) Provisional application No. 62/174,285, filed on Jun. 11, 2015.

(51) Int. Cl. G01N 33/49; H01J 49/00

(52) U.S. Cl. CPC G01N 33/492; H01J 49/0036

(57) ABSTRACT

Provided herein are methods of metabolic kinetic phenotyping based on amino acids, proteins and other metabolites thereof. In the method a solution comprising a plurality of stable isotopes of amino acids is administered to the individual and one or more kinetic parameters of amino acids and the metabolites are calculated in blood samples taken periodically from the individual. The metabolic phenotype is composed from the kinetic parameters. Also provided are methods and kits for identifying a disease, such as chronic heart failure, in a patient using the metabolic parameters.

### CROSS-REFERENCE TO RELATED APPLICATIONS

This application is a non-provisional under 35 U.S.C. §119(e) of provisional application U.S. Ser. No. 62/174,285, filed Jun. 11, 2015, the entirety of which is hereby incorporated by reference.

### BACKGROUND OF THE INVENTION

Field of the Invention

The present invention relates generally to the field of metabolic kinetic phenotyping. More specifically, the present invention is directed to methods of measuring amino acid and corresponding metabolites and its production and disposal by stable isotopes.

Description of the Related Art

Over the past few decades, researchers in the medical field have increasingly realized that personalized or precision therapy is the future of medical industry. It has become clear that the end results of using the same medicine to treat different patients with similar diseases can vary drastically due to an individual's unique genome and fluxome (kinetics of substrates).

Currently, most theories on personalized therapy are based on the genetic makeup for each individual. Generally, a database of genes and their corresponding relation with certain health conditions or responsiveness to drugs are established via extensive clinical research. Then the genetic information of a patient is analyzed through DNA sequencing. The results are subsequently compared with the database, and personalized medicines are then produced based on to the patient's unique genetic information and the established gene-disease database. These medicines are able to specifically target the unique pathological pathways in each individual, maximizing the effectiveness of the medicines. However, current knowledge about the correlations between genetic information and diseases is still very limited. To date, most of the research in this area is constrained to certain types of cancer. A large amount of clinical trials, laboratory work are still needed to fully take advantages of personalized therapy based on genetic information of patients.

Protein and amino acid metabolism patterns are unique for each individual and unique in the response to a disease in this individual. Specific modifications of the “metabolic phenotype” pattern also exist in disease states. Metabolism of amino acids is relatively easy to trace compared to DNA and RNA. By tracing a naturally occurring amino acid, the fluxes of amino acids through metabolic pathways can be qualified in both healthy and diseased conditions, which can provide essential health information for each individual.

Therefore, there is a recognized need for a method of measuring protein breakdown and amino acid metabolic kinetics in humans under both healthy and diseased conditions for use for personalized or precision therapy. The prior art is deficient in this respect. The present invention fulfills this longstanding need and desire in the art.

### SUMMARY OF THE INVENTION

The present invention is directed to a method of phenotyping amino acid and protein metabolic kinetics in an individual. In the method a blood sample is drawn from an individual as a negative control and a solution comprising a plurality of stable isotopes of amino acids or related compounds is administered to into the individual. Blood samples are taken periodically from the individual and the amount of isotopes and the metabolites thereof is measured in each sample; calculating One or more kinetic parameters of amino acids and the metabolites thereof are calculated in the individual and a metabolic phenotype is composed using the kinetic parameters of each amino acid and the metabolites thereof.

The present invention is also directed to a method for identifying a disease of a patient. The assay comprises the steps of creating a metabolic phenotype of individuals with each of the diseases to be tested, creating a metabolic phenotype for one or more healthy individuals and a metabolic phenotype for the patient and comparing the metabolic phenotype of the patient with that of the healthy individuals and individuals with each of the diseases to determine the disease for the patient.

The present invention is further directed to a kit for identifying a disease for an individual based on the individual's protein or amino acid metabolic kinetics. The kit comprises a mixture of isotope labeled amino acids, related compounds or proteins and instructions for using the mixture for phenotyping amino acids or protein metabolic kinetics via the method described herein. The kit also comprises reference phenotypes comprising an amino acids and/or protein metabolic phenotype from healthy individuals, and a set of amino acids and/or protein metabolic phenotypes from individuals with each of the diseases to be tested.

The present invention is directed further to a method for identifying chronic heart failure in an individual in need of such. In the method a solution comprising L-[tau-²H₃]Methyl-Histidine is administered to the individual and to a control subject. A biological sample is taken periodically from the individual and the control subject and measuring the amount of L-[tau-²H₃]Methyl-Histidine and the metabolites thereof is measured in the biological samples of the individual and the control subject. The whole body appearance rates of methyl-histidine in the individual and control subject based on the amount of L-[tau-²H₃]methyl-histidine and the metabolites thereof in the biological samples are calculated. A higher whole body appearance rate of methyl-histidine in the individual compared to the control subject indicates that the individual has chronic heart failure.

Thus, there is a recognized need for ascorbic acid analogs that are antioxidants and nitric oxide donors, but at the same time do not participate in an enzymatic process in a living organism as a substrate or cofactor. The prior art is deficient in these respects. The present invention fulfills this long standing need and desire in the art.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts the calculation models for whole body protein breakdown.

FIG. 2 depicts the experimental design for comparison of primed-continuous protocol with pulse protocol.

FIG. 3 shows examples of plasma tracer/tracee ratio time courses of arterial phenylalanine in a healthy animal and an animal with sepsis.

FIG. 4 illustrates the arterial phenylalanine tracer concentrations after administration of L-[¹⁵N]-Phenylalanine pulse within 60 minutes for healthy and Sepsis animals.

FIGS. 5A-5C show the whole body rate of appearance of phenylalanine in sepsis and healthy pigs using primed-continuous infusion L-[ring-¹³C₆]-Phenylalanine and pulse tracer method (bolus infusion L-[¹⁵N]-Phenylalanine). FIG. 5A depicts the results for primed-continuous infusion L-[ring-¹³C₆]-Phenylalanine. FIG. 5B depicts the results for pulse tracer method (bolus infusion L-[¹⁵N]-Phenylalanine). FIG. 5C illustrates the linear relation between whole body appearance rates of phenylalanine for primed-continuous infusion and pulse tracer method.

FIGS. 6A-6B depict the intracellular protein break down (PB) data obtained from compartmental modeling (pule method). FIG. 6A shows whole body intracellular protein breakdown for healthy and sepsis pigs. FIG. 6B illustrates the correlation between intracellular protein breakdown and whole body rate of appearance (WbRₐ).

FIGS. 7A-7F depict the results of the whole body rate of appearance for each amino acids tested for groups of humans including young healthy adults, older healthy adults, individuals with chronic heart failure and an individual with chronic obstructive pulmonary disease. FIG. 7A shows the result of the whole body rate of appearance (WbRₐ) of glycine in the four groups of individuals comprising young healthy adults, older healthy adults, individuals with chronic heart failure and an individual with chronic obstructive pulmonary disease. FIG. 7B shows the result of the whole body rate of appearance (WbRₐ) of taurine in the four groups of individuals. FIG. 7C shows the result of the whole body rate of appearance (WbRₐ) of glutamine in the four groups of individuals. FIG. 7D shows the result of the whole body rate of appearance (WbRₐ) of leucine in the four groups of individuals. FIG. 7E shows the result of the whole body rate of appearance (WbRₐ) of citrulline in the four groups of individuals. FIG. 7F shows the result of the whole body rate of appearance (WbRₐ) of methyl-histidine in the four groups of individuals.

FIG. 8 depicts the whole body rate of appearance and related interconversions for the metabolites of the isotope labeled amino acids in individuals under four different health conditions: healthy young adults, healthy older adults, chronic heart failure and chronic obstructive pulmonary disease.

### DETAILED DESCRIPTION OF THE INVENTION

As used herein in the specification, “a” or “an” may mean one or more.

As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

In one embodiment of the present invention, there is provided a method of phenotyping amino acids and protein metabolic kinetics in an individual comprising the steps of drawing a blood sample from an individual as a negative control; administering a solution comprising a plurality of stable amino acids isotopes or related compounds to the individual; taking blood samples periodically from the individual; measuring the amount of isotopes and the metabolites thereof in each sample; calculating one or more kinetic parameters of amino acids and the metabolites thereof in the individual; and composing metabolic phenotypes using the kinetic parameters of each amino acid and the metabolites thereof.

In this embodiment, the solution comprising a plurality stable isotope of amino acids is administered to the individual in a pulsed pattern. Representative stable isotopes of amino acids are, including but not limited to, L-[ring-²H₅]Phenylalanine, L-[U-¹³C₉¹⁵N]Tyrosine, L-[²H₃]Leucine, [1-¹³C]KIC, L-[tau-²H₃]Methyl Histidine, L-[2-²H-OH]Proline, [²H₂]Glycine, L-[guanidine-¹⁵N₂]Arginine, L-[5-¹³C-²H₂]Citrulline, L-[5-¹⁵N]Glutamine, L-[1,2-¹³C₂]Glutamate, ¹³C-Urea, L[1,2-¹³C₂]Taurine, L-[¹⁵N₂]Tryptophan or a combination thereof. In this embodiment, the solution of stable isotopes of amino acids is administered intravenously. Typically, the blood samples are taken periodically at a frequency of about every 15 minutes for about 3 hours.

The amino acids and the metabolites thereof may be measured using an appropriate apparatus, such as a mass spectrometry. Representative examples of the metabolites are, including but not limited to citrulline from arginine, arginine for citrulline, tyrosine from phenylalanine, KIC from leucine, leucine from KIC, HMB from leucineor a combination thereof. Also, the representative examples of the kinetic parameters are, including but not limited to, whole body rate of appearance, intracellular appearance, protein synthesis, protein breakdown, nitric oxide production, arginine de novo production, or a combination thereof. The kinetic parameters are calculated using a non-compartmental or compartmental model.

In another embodiment of the present invention, there is provided a method for identifying a disease of a patient comprising a) creating a metabolic phenotype of each of the diseases to be tested comprising drawing a blood sample from the healthy individual as a negative control; administering a solution comprising a plurality stable isotopes of amino acids or related compounds into the individual; taking blood samples periodically from the individual; measuring the amount of isotopes and the metabolites thereof in each sample and calculating one or more kinetic parameters of amino acids and the metabolites thereof for the individual; b) creating a metabolic phenotype for one or more healthy individuals using the same method as step a); c) comparing the phenotypes from step a) with the phenotype from step b) to record the variance of the metabolic kinetics between healthy individuals and individuals with each disease; d) creating a metabolic phenotype for said patient using the same method as step a); and e) identifying the type of disease of said patient based on the metabolic phenotype from step d) and said variance from step c).

In this embodiment, the solution containing four or more stable isotopes of amino acids. Representative stable isotopes of amino acid are, including but not limited to, L-[ring-²H₅]Phenylalanine, L-[U-¹³C₉, ¹⁵N]Tyrosine, L-[²H₃]Leucine, [1-¹³C]KIC, L-[tau-²H₃]Methyl Histidine, L-[2-²H-OH]Proline, [²H₂]Glycine, L-[guanidine-¹⁵N₂]Arginine, L-[5-¹³C-²H₂]Citrulline, L-[5-¹⁵N]Glutamine, L-[1,2-¹³C₂]Glutamate, ¹³C-Urea, L-[1,2-¹³C₂]Taurine, L-[¹⁵N₂]Tryptophan or a combination thereof. Also the solution of stable isotope of amino acids is administered intravenously. The blood samples are taken periodically at a frequency of about every 15 minutes for about 3 hours.

The amino acids and the metabolites thereof are measured using a mass spectrometry. The representative examples of the metabolites are, including but not limited to, to citrulline from arginine, arginine for citrulline, tyrosine from phenylalanine, KIC from leucine, leucine from KIC, HMB from leucine or a combination thereof. In this embodiment, the representative examples of the kinetic parameters are, including but not limited to, whole body rate of appearance, intracellular appearance, protein synthesis, protein breakdown, nitric oxide production, arginine de novo production, or a combination thereof. Also, the kinetic parameters are calculated using a non-compartmental or compartmental model. Representative examples of diseases to be tested are, but not limited to, heart failure, chronic obstructive pulmonary disease, cancer, diabetes, obesity, sepsis, liver cirrhosis or a combination thereof.

In yet another embodiment of the present invention, there is provided a kit for identifying a disease of an individual based on protein or amino acid metabolic kinetics. The kit comprises a mixture of isotope labeled amino acids or proteins; instructions for using the mixture for phenotyping amino acids or protein metabolic kinetics via the method as described supra; and reference phenotypes comprising an amino acids and/or protein metabolic phenotype from healthy individuals, and a set of amino acids and/or protein metabolic phenotypes from individuals with each of the diseases to be tested. In this embodiment, the disease to be tested comprises chronic heart failure, chronic obstructive pulmonary disease, cancer, obesity, sepsis, liver cirrhosis, or a combination thereof.

In yet another embodiment of the present invention, there is provided a method for identifying chronic heart failure in an individual in need of such comprising the steps of administering a solution comprising L-[tau-²H₃]Methyl-Histidine to the individual and to a control subject; taking a biological sample periodically from the individual and the control subject; measuring the amount of L-[tau-²H₃]Methyl-Histidine and the metabolites thereof in the biological samples of said individual and said control subject; and calculating whole body appearance rates of methyl-histidine in said individual and control subject based on said amount of L-[tau-²H₃]methyl-histidine and the metabolites thereof in the biological samples where a higher whole body appearance rate of methyl-histidine in the individual compared to the control subject indicates that the individual has chronic heart failure.

The solution comprising L-[tau-²H₃]methyl-histidine is administered in a pulsed pattern and may be adminstered intravenously. Also, the biological sample is blood or plasma. The biological samples may be taken periodically at a frequency of about every 15 minutes for about 3 hours. The enrichment of L-[tau-²H₃]methyl-histidine is used to calculate the production of L-methyl-histidine. In this embodiment the amount of L-[tau-²H₃]methyl-histidine and the metabolites thereof are measured using mass spectrometry. The whole body rates of appearance are calculated using a non-compartmental or compartmental model.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Subjects for the Experiment

Female Yorkshire cross/domestic pigs (20-25 kg BW) were used in the experimental studies. The pigs were housed in steel pens (2 m×3 m) in a controlled housing facility with large animal cubicle at room temperature 22-24° C. with 12 hours light-dark cycle. The pigs were fed with standardized food 1 kg/day (Harlan Teklad Vegetarian Pig/Sow Grower)) and provided water ad libitum.

Animals received catheters and a jejunal stoma during a surgical procedure. During midline laparotomy, catheters were placed into the abdominal aorta for blood sampling, and in the caval vein for administering post-surgery medication and experiment related tracer infusions. A second arterial catheter was placed to monitor mean arterial blood pressure (MAP). A Swan ganz catheter (5 Fr, #132F5, Edwards life sciences, Irvine, Calif., USA) was placed via the right jugular vein to monitor mean pulmonary blood pressure (MPAP). Both preoperative and postoperative cares were standardized. During the recovery period (7-10 days) animals were accustomed to a small movable cage (0.9×0.5×0.3 m). The experiments were performed in this cage on awake animals. This study was approved by the animal experiment ethics committee of University of Arkansas Medical Sciences.

Pseudomonas aeruginosa

For the induction of sepsis, a live Pseudomonas aeruginosa (PM) human strain is used (IRS 12-4-4, Shriners burns institute, University of Texas Medical Branch, Galveston). Originally, this PM strain was isolated from a burnt patient at Brook Army Medical Center in San Antonio, Tex. Based on pilot virulence experiment, 10⁹ Colony Forming Units per hour (CFU/hour) in a volume of 1 ml 0.9% NaCl solution was needed to obtain similar cardiovascular responses and hemodynamic variables with characteristics of severe hyperdynamic sepsis. Hemodynamics was continuously monitored to ensure that the hyperdynamic state was kept in the expected ranges for severe sepsis (body temp increase of 2-3° C., respiratory rate increased, MPAP increased but <35 mmHg, heart rate increased but <200 BPM).

### Example 2

### Experimental Design

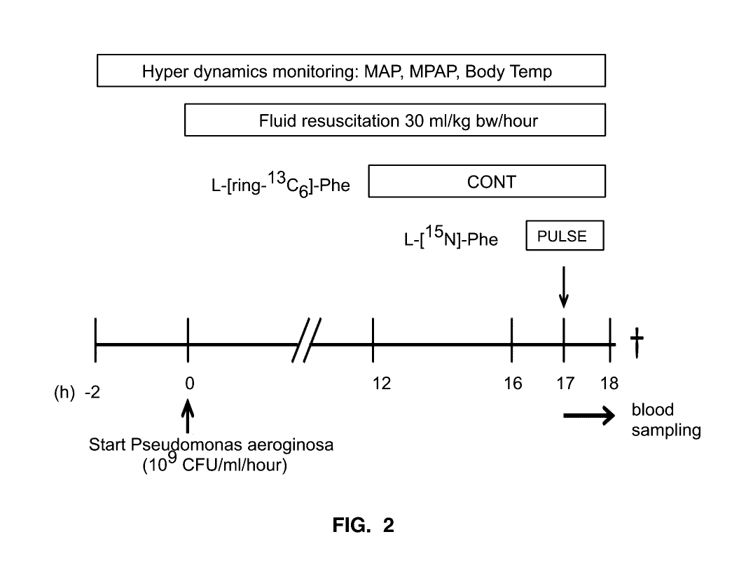

The experiment started after a recovery period of 7-10 days. Four hours after the last food intake (half of the daily amount: 0.5 kg), animals were selected from the Sepsis group or the Healthy group in a randomized fashion. As illustrated in FIG. 2, the basal monitoring blood pressures were monitored in the pre-septic period (T=−2 h−0 h). At T=0 h, sepsis was induced to the sepsis group by continuous infusion of Pseudomonas aeruginosa (PM, 10⁹ CFU/ml/hour), while the Healthy group received an equal volume of 0.9% NaCl solution. Fluid resuscitation (30 ml/kg bw/hour) was also started at T=0 h and hemodynamics were monitored continuously. The results for the primed-continuous tracer protocol (PC) and the pulse tracer protocol were compared between 17 and 18 hours after the start of Pseudomonas aeruginosa. At t=18 h, the pigs were euthanized with 125 mg/k pentobarbital sodium and 16 mg phenytoin sodium (Euthanasol®).

### Example 3

### Protocols for Infusion and Sampling

### Stable Isotopes

Two stable isotopes of Phe: L-[ring-¹³C₆]-Phe and L-[¹⁵N]-Phe (Cambridge Isotopes, Andover, Mass.) were used as tracers to study whole body rate of appearances of Phe (WbRₐ) with two tracer infusion protocols. Phe has been used to determine whole body protein breakdown. Previously studies were conducted based on the prime amount and tracer infusion rates. For the primed-continuous infusion protocol (PC), L-[ring-¹³C₆]-Phe was used. The prime (1.58 μmol/kg bw) and infusion (4.32 μmol/kg bw/hour) was given respectively in a volume of 2 ml/kg bw and 2 ml/kg bw/hour. It started 12 hours after the start of Pseudomonas aeruginosa, which is also 5 hours before the pulse protocol (PULSE). The L-[¹⁵N]-Phe (26.3 μmol/kg bw in a volume 0.5 ml/kg bw) was used for the pulse protocol. All tracers are given via the central caval vein catheter.

### Blood Sampling and Sample Processing

Blood samples were taken and directly cooled on ice. The blood samples were processed within one hour. For amino acid concentration and enrichment analysis, heparinized blood was centrifuged at 4° C., 8000 G for 5 minutes. Then, 250 μL plasma with 25 μL tri-chloroacetic acid solution (TCA, 50% w/v) was deproteinized and finally snap frozen in liquid nitrogen and stored at −80° C.

### Example 4

### Determination of Amino Acid Concentration and Enrichments

Amino acid isotope concentrations and amino acid enrichments (tracer-to-tracee ratios, TTR) were determined using a fully automated LC-ESI-MS system (QTrap 5500 MS (AB Sciex, Foster City, Calif., USA) with ExpressHT Ultra LC (Eksigent Div., AB Sciex, Foster City, Calif., USA). Supernatant (20 μl) of centrifuged tri-chloroacetic acid (TCA) solution deproteinized plasma was added to a 0.1 N hydrochloric acid containing internal standard (20 μl) with the stable isotopomer L-[U-¹³C₉]-Phe and L-[D₈]-valine (for internal response check) for concentration measurements. For tracer-to-tracee ratios measurement, only the internal standard with L-[D₈]-valine was added. Within 3 days before the LC-ESI-MS analysis, plasma samples were derivatized with internal standard and external standards at concentrations within the physiological range and internal standard containing enriched external standards in the range of expected TTRs (calibration curve for TTRs) with 9-fluorenylmethoxycarbonyl (Fmoc). After neutralization, 160 nL of the solution were injected into a micro LC column 0.5×100 mm HALO C18, 2.7 um, 90A pores (ABsciex, Foster City, Calif., USA), and kept at 35° C. Analytes was eluted with a segmentally linear gradient from 35% to 85% acetonitrile in water supplemented with ammonium acetate to 10 μM and 5% isopropanol. Electrospray triple quadrupole tandem mass spectrometry was adjusted in multiple reactions monitoring mode for detection. The Fmoc amino acid derivatives were fragmented in the collision cell for the detection of either free aminoacyl anions or a fragment coming from the Fmoc derivative to have the highest sensitivity. The mass analyses for phenylalanine, its tracers and internal standards were simultaneously conducted. The mass signal areas were calculated to enable TTR or tracee concentrations calculations. The mass signal of the L-[D₈]-valine was used as the quality control of the Fmoc derivatization procedure.

### Example 5

### Calculation of Phenylalanine Tracee or Tracer Concentration and Tracer-to-Tracee Ratios

To calculate concentrations, the tracee signals of the samples and the external standards were normalized with their internal L-[¹³C₉]-Phe standards. The plasma tracee concentration was determined using the calibration curve of the external standards. The plasma TTR was calculated as tracer signal divided by tracee signal and corrected for background. A calibration curve of tracer containing external standards was used to calculate the tracer concentration in the infusates.

Calculation WbRₐ with Prime-Constant and Pulse Protocol

The whole body rates of Phe appearances (WbRₐ) into the circulation in a post-absorptive (patho-) physiological state were calculated using a non-compartmental model for the prime-constant group and a compartmental model for the pulse group. The results from the two models are compared.

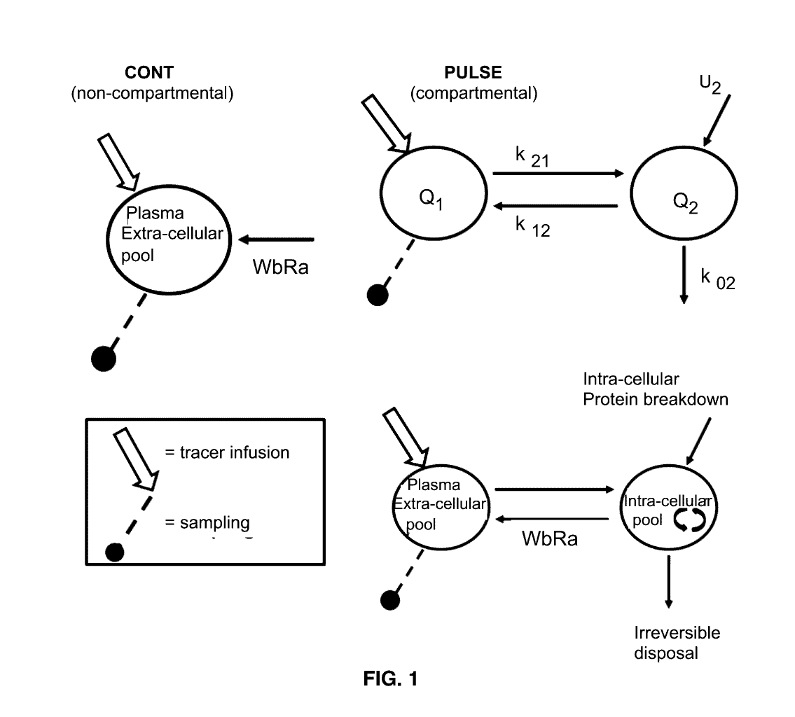

For prime-constant: As illustrated in FIG. 1, the WbRₐ is derived from equation (1) by using the L-[13C6]-Phe isotope. The tracer infusion rate (I) is divided by tracer to tracee ratio in arterial plasma (TTRa).

WbRₐ=1/TTR Eq. (1)

For pulse: Non-compartmental analysis in GraphPad Prism® (version 6) is used to perform curve fitting of the exponential decay curve. The results showed that the data fit best with a 2-compartment model, which corresponds to the expectations for essential amino acids. Subsequently, computational multi-exponential curve fitting in SAAM® II (Version 2.2: The Epsilon Group; Charlottesville, Va.) was used to calculate the k values and pool sizes in a two compartmental model. The k values were converted to whole-body rate of appearance (WbRₐ) or intracellular production. The proportionality constants (k₁₂, k₂₁, k₀₂) and the plasma pool size (Q₁) with SAAM II® were obtained using the equations of the individual curves. Assuming the measurements were done in a physiological steady state, meaning no net loss or production of Phe tracee in Q₁ during the experimental period, the flux (F) to Q₁ from Q₂ (F₁₂) is calculated as:

Flux=F₁₂=F₂₁=k₂₁×Q₁ Eq. (2)

The size of the intracellular pool (Q₂) is calculated with:

Intracellular pool=Q₂=F₁₂/k₁₂ Eq. (3)

The irreversible loss from the intracellular pool (F₀₂):

Irreversible loss=F₀₂=k₀₂×Q₂ Eq. (4)

In a physiological steady state, irreversible loss is equal to appearance of phenylalanine in the intracellular pool. Assuming that the appearance of Phe in the intracellular pool is coming from protein breakdown, the intracellular protein breakdown is determined by Equation (5):

Intracellular protein breakdown=F₂₀=F₀₂ Eq. (5)

The fraction of the amount of phenylalanine coming from protein breakdown that will appear in the extracellular pool (Q₁) is the amount that is not irreversible lost:

WbRₐ=F₂₀*(1−F₀₂/F₂₀+F₂₁)) Eq. (6)

### Statistical Analyses

Results are presented as means±standard error means. Graphpad Prism® (version 6) was used for statistics. Levels of significance was set to p<0.05. To determine physiological tracee or tracer Phe steady state during the experimental period, linear regression was used to determine if the slope of the best fitted line was different from zero. No difference from zero indicates steady state. To compare data between the Healthy and Sepsis group, an unpaired t-test was used. To compare physiological relevant models/parameters, a Pearson correlation test was used. Best-fitted line that describes the relation between both models/parameters was done with linear regression. A shared fitted line was determined when no significant differences were observed between the Healthy and the Sepsis fitted line.

### Example 6

### Characteristics and Validation of Tracer Models

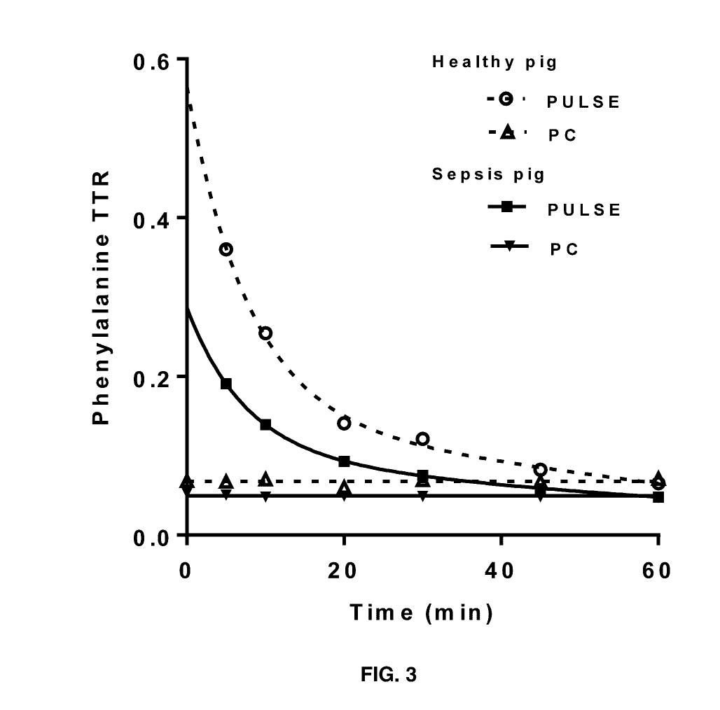

FIG. 3 shows the enrichment (TTR versus time) curve for a pulse of L-[¹⁵N]-Phe and the primed-continuous infusion enrichment of L-[¹³C₆]-Phe for both sepsis and healthy animals. Overall, the enrichment follows a exponential decay. The coefficient of determination (R square): 0.9991±0.0002 (Healthy) and 0.9991±0.002 (Sepsis). The results also show all the tracer reached steady states during the experimental period for all the tested animals.

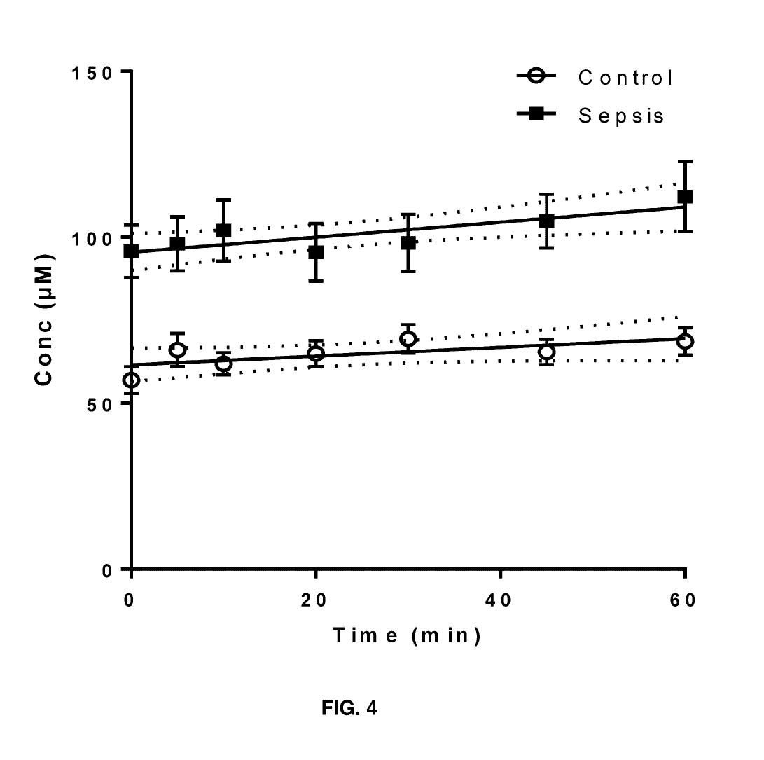

FIG. 4 shows the arterial tracee Phe concentrations during the experimental period. Both experimental groups (Healthy and Sepsis) showed tracee steady states, which means not net loss or production of Phe tracee in the plasma and extracellular pool. This is essential for the 2-compartmental model. FIG. 4 shows that plasma Phe concentration was increased in the sepsis animals (median over the experimental period, Healthy: 64.6±4.0 μM; Sepsis 113±8.3 μM; p=0.0002).

The results of the accuracy analysis for compartmental model parameters (Table 1) show that all coefficients of variation were below 100%, indicating that the model should be accepted.

TABLE 1
Accuracy of compartmental model

| | Healthy | | | Sepsis | | |
|---|---|---|---|---|---|---|
| | Mean | Mean | Mean | Mean | Mean | Mean |
| Parameter | Value | SD | VC % | Value | SD | VC % |
| Q₁ (μmol) | 1121 | 49 | 4.1 | 1655 | 37 | 2.5 |
| k₀₂ (min⁻¹) | 0.033 | 0.005 | 14 | 0.028 | 0.003 | 11 |
| k₁₂ (min⁻¹) | 0.051 | 0.020 | 26 | 0.044 | 0.006 | 14 |
| k₂₁ (min⁻¹) | 0.115 | 0.019 | 14 | 0.105 | 0.008 | 7.0 |

Mean variations of parameters generated from the fitting of individual decay curves with computer software SAAM II: Q₁ is plasma pool size; k₀₂, k₁₂, k₂₁ are proportionality constants.

### Example 7

### Whole Body Rate of Appearance (WbRₐ) Comparisons

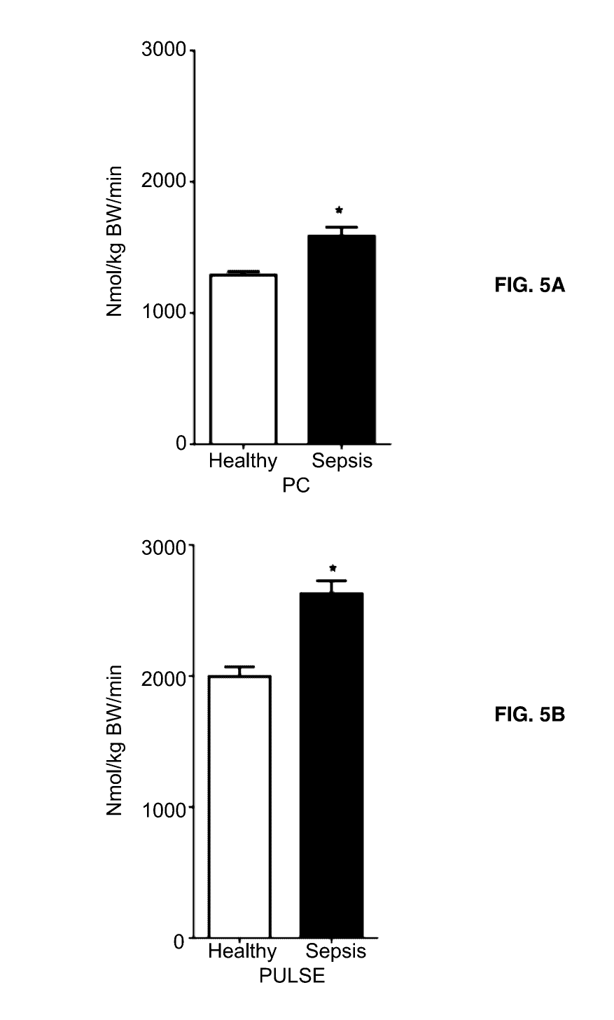

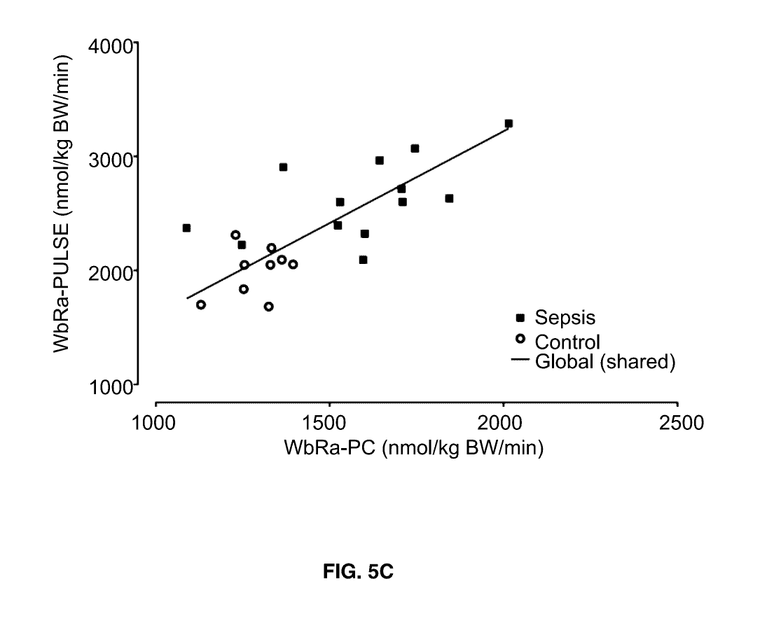

The WbRₐ's for both healthy and septic animals are used to determine whether both prime-constant infusion and pulse trace were able to indicate the changes in protein breakdown. FIGS. 5A-5C show that WbRₐ of Phe was higher in sepsis group with both methods (PC: p=0.003; PULSE: p=0.0001), albeit that WbRₐ were about 1.6 times higher for the Pulse method than the prime-constant infusion. The results indicate that the correlation between the prime-constant infusion and pulse method are statistically significant (r=0.732, p<0.0001).

### Example 8

### Comparison of WbRₐ with Intracellular Protein Breakdown

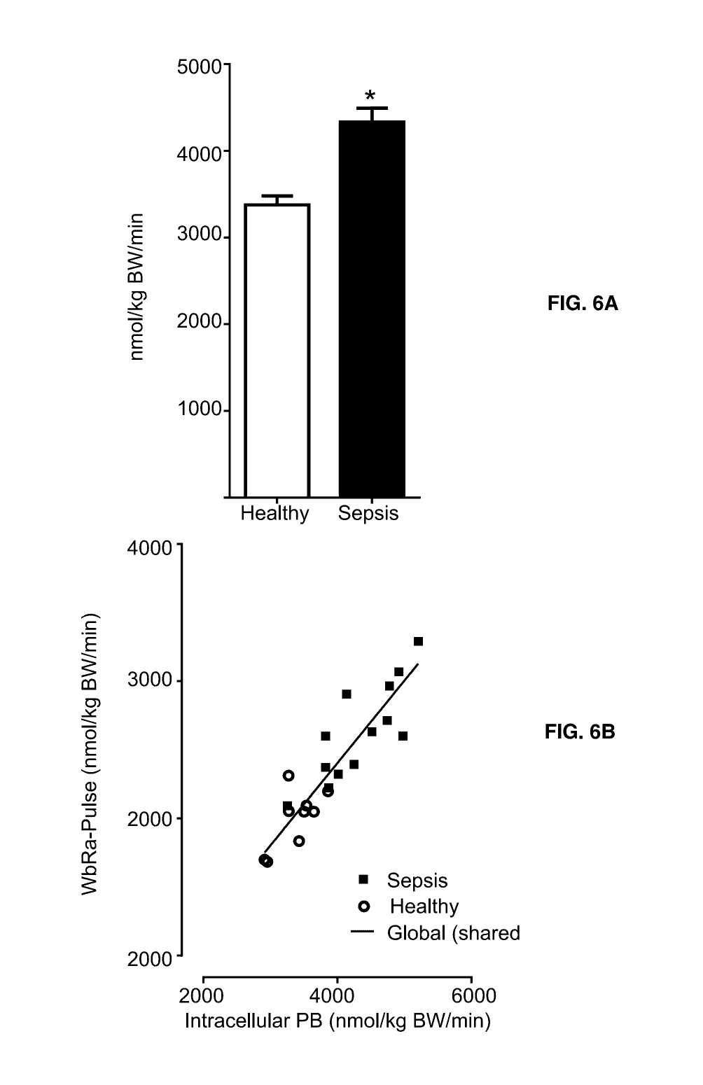

Both WbRₐs derived based on Phe in the extracellular pool using prime-constant infusion and pulse method have been compared with the intracellular protein breakdown (PB). The results in FIGS. 6A-6B show that the correlation between WbRₐ derived using pulse method and intracellular protein breakdown was statistically significant (r=0.897, p<0.0001) in the sepsis group. The intracellular protein breakdown was 1.7 times higher than the WbRₐ using pulse method.

Both extracellular and intracellular Phe pools were increased in sepsis) and are statistically highly correlated (r=0.802: p<0.001). The intracellular pool is 2.2 times larger than the extracellular pool. The extracellular Phe pool size also relates well to the plasma Phe concentration (r=0.613; p=0.002).

An increase in Phe flux between extra- and intracellular pools and an increase in irreversible loss was also measured (Table 2) using the pulse method. No changes are seen in the fractional release, uptake and irreversible loss of Phe in the intracellular pool.

TABLE 2
Extra whole body metabolic information
obtained with PULSE model

| Parameter | Unit | Healthy | Sepsis | p-value |
|---|---|---|---|---|
| EC Phe pool | μmol/kg BW | 44 ± 2.2 | 63 ± 5.5 | 0.002 |
| IC Phe pool | μmol/kg BW | 105 ± 8.0 | 157 ± 8.2 | 0.0003 |
| Flux | μmol/kg | 5.02 ± 0.40 | 6.86 ± 0.42 | 0.006 |
| | BW/min | | | |
| Irr. loss | μmol/kg | 3.38 ± 0.10 | 4.33 ± 0.16 | 0.0002 |
| | BW/min | | | |
| Frac. irr. loss | %/min | 3.33 ± 0.21 | 2.83 ± 0.15 | 0.065 |
| IC Phe | %/min | 5.09 ± 0.69 | 4.43 ± 0.25 | 0.316 |
| release | | | | |
| IC Phe | %/min | 11.5 ± 0.71 | 10.6 ± 0.76 | 0.423 |
| uptake | | | | |

Mean values of parameters are obtained and are calculated from the fitting of individual decay curves. EC Phe pool is extracellular Phe pool (Q₁); IC Phe pool is intracellular Phe pool (Q₂); Flux is flux of Phe between intra- and extracellular pool (F₁₂, F₂₁); Irr. loss is irreversible loss of Phe from the intracellular pool (F₀₂); Frac. irr. loss is fractional irreversible loss of Phe from the intracellular pool (k₀₂); IC Phe release is fractional intracellular release of Phe to extracellular pool (k₁₂); IC Phe uptake is fractional intracellular uptake of Phe from extracellular pool (k₂₁). Values are expressed as mean±SEM. Healthy n=9; Sepsis n=13. Statistics: unpaired t-test.

### Example 9

### Study of Metabolic Kinetic Phenotyping Using the Pulse Method on Humans

The pulse method was utilized for studying human metabolic kinetic phenotyping. In this study, 10 young healthy adults and 14 older adults, 11 adults with chronic heart failure, 12 adults with chronic obstructive pulmonary disease were selected. A set of stable isotopes (15 different isotopes) was administered using the pulse method. Isotopes and corresponding metabolites (23 different isotopomers) were measured.

TABLE 3
The stable isotopes used for human study

| Stable Isotope | What is measured (in addition to WbRa) |
|---|---|
| 2L-[ring-²H₅]Phenylalanine | Protein Synthesis-Breakdown-Net |
| L-[U—¹³C₉,¹⁵N]Tyrosine | protein synthesis/breakdown |
| L-[²H₃]Leucine | Leucine trans- and re-amination |
| [1-¹³C]KIC | |
| L-[tau-²H₃]Methyl Histidine | Myofibrillar protein breakdown |
| L-[2-²H—OH]Proline | Collagen protein breakdown |
| [²H₂]Glycine | GSH synthesis |
| L-[guanidine-¹⁵N₂]Arginine | NO production, Arg de novo |
| L-[5-¹³C—²H₂]Citrulline | production, Arginase activity |
| L-[5-¹⁵N]Glutamine | Glutamine trans- and re-amination |
| L-[1,2-¹³C₂]Glutamate | |
| ¹³C-Urea | Protein breakdown |
| L-[1,2-¹³C₂]Taurine | Taurine production |
| L-[¹⁵N₂]Tryptophan | Serotonin production |

### Experimental Protocol

Subjects were ordered to fast (food and drink other than water) from 10 μm±2 h onwards the night before the test day. The next morning, the body weight of each of the subjects is recorded. Body composition (whole body fat mass and fat-free mass) of each of the subjects was assessed by dual-energy X-ray absorptiometry. The subjects then lie in a supine or an elevated position for 3 hours. Before the administration of the pulsed isotopes, a venous blood sample was collected for the subject to measure the natural enrichment of amino acids and keto-acids as the control. A catheter was then placed in a superficial vein of a hand of the subject to infuse a solution containing the “tracer” comprising multiple stable isotopes to investigate simultaneous production and breakdown rate of proteins and behavior of multiple amino acids and keto-acids, and to study specific substrate kinetics related to corresponding metabolic pathways. After the administration of the pulsed isotopes, the same catheter was used for arterialized-venous blood draws while placing the subjects hand in a thermostatically controlled hot box at 55° C. Arterialized-venous blood was sampled every 15 minutes in the 3 hours period. The total amount of blood drawn from each subject was about 70 ml.

The blood samples were put in Li-heparinized tubes, immediately put on ice and instantly frozen and stored at −80° C. until further analyses. All the samples were analyzed using L-ESI-MS as described supra. The calculations of the whole body rates of appearance of each amino acid, related metabolites and interconversions were conducted using the non-compartmental and compartmental model described supra. The statistical analysis was done with the same method described in Example 5.

### Results of the Whole Body Rate of Appearance

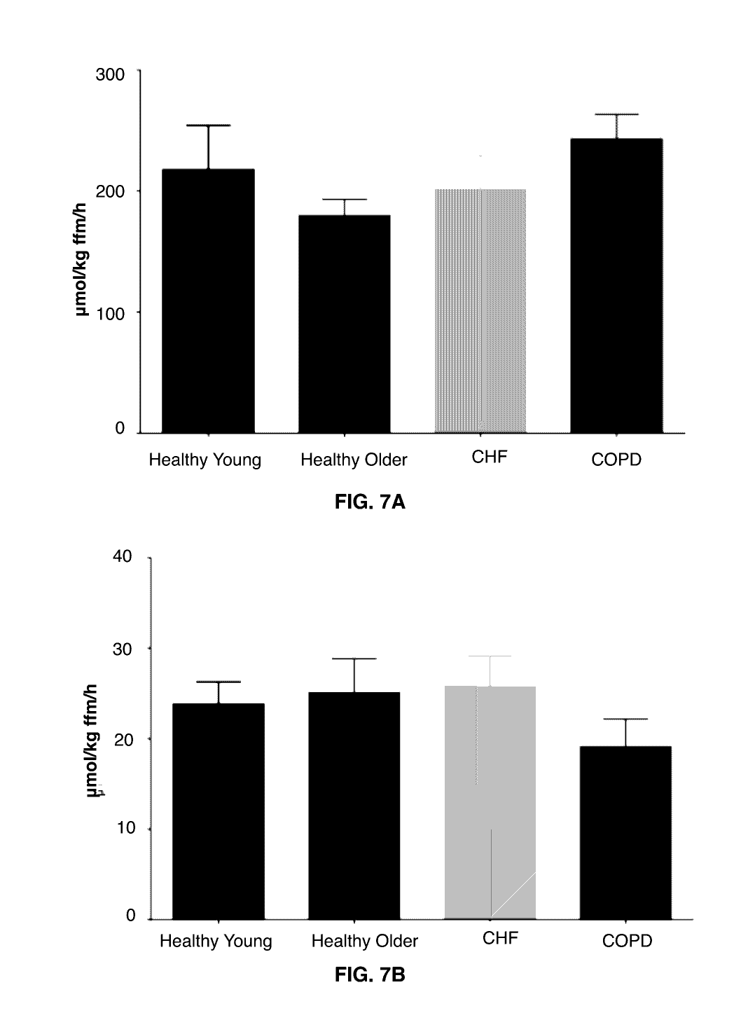

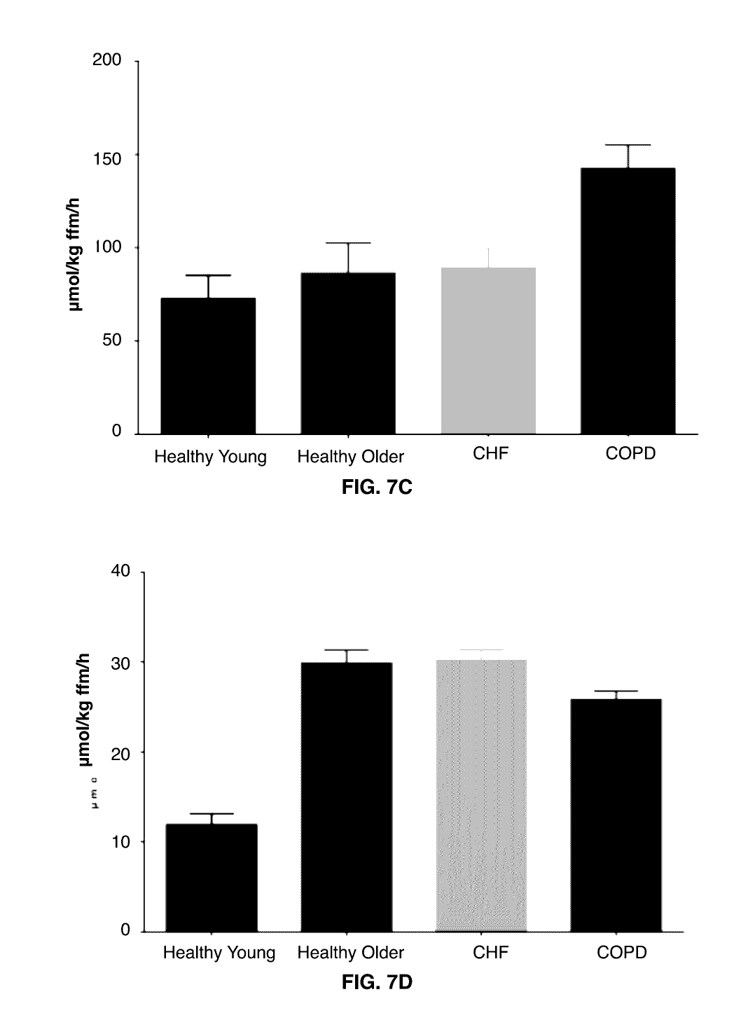

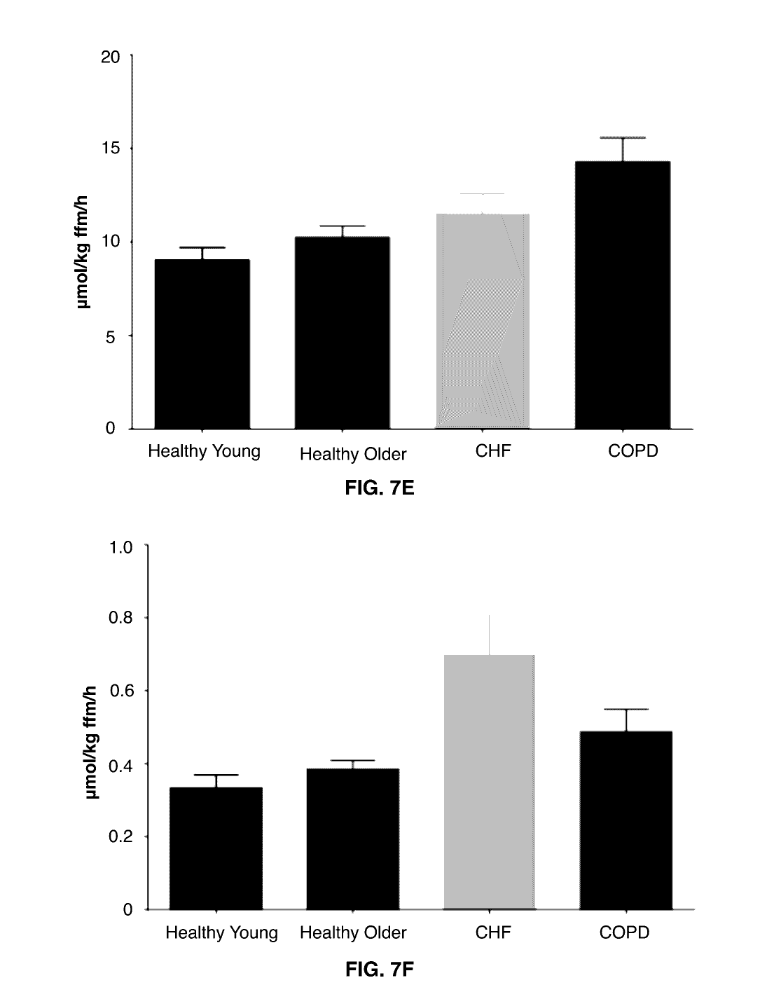

FIGS. 7A-7F show the results of the amino acid whole body rate of appearance (WbRₐ). FIG. 7A depicts that for glycine, the subjects with chronic obstructive pulmonary disease had the highest whole body rate of appearance of about 220 μmol/kg ffm/hour. The healthy older adults showed the lowest whole body rate of appearance of about 180 μmol/kg ffm/hour.

FIG. 7B shows that the subjects with chronic obstructive pulmonary disease had the lowest WbRₐ for taurine. The WbRₐ's for healthy young adults, healthy older adults and individuals with chronic heart failure were not significantly different from each other. For glutamine as shown in FIG. 7C, the individuals with chronic obstructive pulmonary disease have the highest WbRₐ, while the WbRₐ for healthy young adults is the lowest.

For leucine as shown in FIG. 7D, the healthy young adults have the lowest WbRₐ. The WbRₐ's for healthy older adults and individuals with chronic heart failure are the same, which is higher than WbRₐ for individuals with chronic obstructive pulmonary disease.

As shown in FIG. 7E, the healthy young adult has the lowest WbRₐ for citrulline while the individuals with chronic obstructive pulmonary disease has the highest WbRₐ for citrulline. The WbRₐ of citrulline for individuals with chronic heart failure is higher than the healthy older adults.

For the results of methyl-histidine, FIG. 7F shows that the individual with chronic heart failure has the highest WbRₐ of about 0.75 μmol/kg ffm/hour. The individuals with chronic obstructive pulmonary disease have the second highest level of WbRₐ of about 0.5 μmol/kg ffm/hour. The WbRₐ's of methyl-histidine for healthy young adults and healthy older adults do not shown any significant difference.

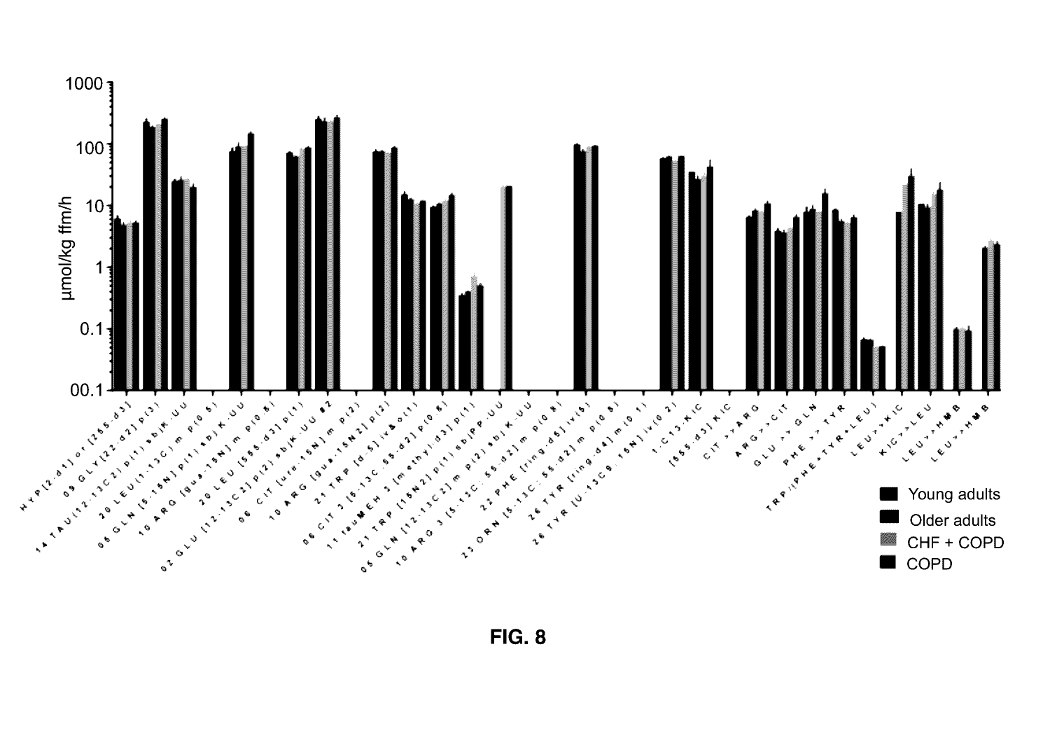

The results of the whole body rates of appearance for the metabolites of the amino acids are presented in FIG. 8. There are 23 types of metabolites analyzed in the experiments. The results indicate that the WbRₐ's for each of the metabolites varies with individuals of different health conditions (young healthy adults, older healthy adults, individuals with chronic heart failure and individuals with chronic obstructive pulmonary disease). Especially for 21 TRP [15N2]p(1)sbjPP-UU, the WbRₐ's for both young and healthy adults are less than 0.01 μmol/kg ffm/hour, while the individuals with chronic heart failure and chronic obstructive pulmonary disease have the WbRₐ of about 20.01 μmol/kg ffm/hour. For Leu>>HMB, WbRₐ for young healthy adults is less than 0.01 μmol/kg ffm/hour, while subjects with other health conditions shows a WbRₐ of about 3 μmol/kg ffm/hour.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention.

What is claimed is:

1. A method of phenotyping amino acids and proteins metabolic kinetics in an individual comprising the steps of:
drawing a blood sample from an individual as a negative control;
administering a solution containing four or more stable isotopes of amino acids into said individual;
taking blood samples periodically from said individual;
measuring the amount of isotopes of amino acids and the metabolites of amino acids thereof in each sample;
calculating one or more kinetic parameters of amino acids or related compounds and the metabolites thereof in said individual; and
composing a metabolic phenotype using said kinetic parameters of each amino acid and the metabolites thereof.

2. The method of claim 1, wherein said solution is administered in a pulsed pattern.

3. The method of claim 1, wherein said stable isotope comprises L-[ring-²H₅]Phenylalanine, L[U-¹³C₉, ¹⁵N]Tyrosine, L-[²H₃]Leucine, [1-¹³C]KIC, L-[tau-²H₃]Methyl Histidine, L-[2-²H-OH]Proline, [²H₂]Glycine, L-[guanidine-¹⁵N₂]Arginine, L-[5-¹³C-²H₂]Citrulline, L-[5-¹⁵N]Glutamine, L-[1,2-¹³C₂]Glutamate, ¹³C-Urea, L[1,2-¹³C₂]Taurine, L-[¹⁵N₂]Tryptophan or a combination thereof.

4. The method of claim 1, wherein said solution of stable isotopes is administered intravenously.

5. The method of claim 1, wherein said blood samples are taken periodically at a frequency of about every 5 to 15 minutes for about 3 hours.

6. The method of claim 1, wherein said amino acids and the metabolites thereof are measured using mass spectrometry.

7. The method of claim 1, wherein said kinetic parameter comprises whole body rate of appearance, intracellular appearance, protein synthesis, protein breakdown, nitric oxide production, arginine de novo production or a combination thereof.

8. The method of claim 1, wherein said kinetic parameters are calculated using a non-compartmental or compartmental model.

9. The method of claim 1, wherein said metabolites are citrulline, arginine, tyrosine, KIC, leucine, HMB, or a combination thereof.

10. A method for identifying a disease of a patient, comprising:
a) creating a metabolic phenotype of individuals with each of the diseases to be tested comprising:
drawing a blood sample from said individual as a negative control;
administering a solution comprising four or more stable isotopes of amino acids into said individual;
taking blood samples periodically from said individual;
measuring the amount of isotopes and the metabolites thereof in each sample; and
calculating one or more kinetic parameters of amino acids and the metabolites thereof for said individual;
b) creating a metabolic phenotype for one or more healthy individuals using the same method as step a);
c) comparing the phenotypes from step a) with the phenotype from step b) to record the variance of the metabolic kinetics between healthy individuals and individuals with each disease;
d) creating a metabolic phenotype for said patient using the same method as step a); and
e) identifying the type of disease of said patient based on the metabolic phenotype from step d) and said variance from step c).

11. The method of claim 10, wherein said solution containing four or more stable isotopes of amino acids is administered in a pulse pattern.

12. The method of claim 10, wherein said stable isotope comprises L-[ring-²H₅]Phenylalanine, L-[U-¹³C₉, ¹⁵N]Tyrosine, L-[²H₃]Leucine, [1-¹³C]KIC, L[tau-²H₃]Methyl Histidine, L-[2-²H-OH]Proline, [²H₂]Glycine, L-[guanidine-¹⁵N₂]Arginine, L-[5-¹³C-²H₂]Citrulline, L-[5-¹⁵N]Glutamine, L-[1,2-¹³C₂]Glutamate, ¹³C-Urea, L[1,2-¹³C₂]Taurine, L-[¹⁵N₂]Tryptophan or a combination thereof.

13. The method of claim 10, wherein said solution is administered intravenously.

14. The method of claim 10, wherein said blood samples are taken periodically at an interval of 15 minutes for 3 hours.

15. The method of claim 10, wherein said amino acids and the metabolites thereof are measured using a mass spectrometry.

16. The method of claim 10, wherein said kinetic parameter comprises whole body rate of appearance, intracellular appearance, protein synthesis, protein breakdown, nitric oxide production, arginine de novo production, or a combination thereof.

17. The method of claim 10, wherein said kinetic parameters are calculated using a non-compartmental or compartmental model.

18. The method of claim 10, wherein said disease comprises chronic heart failure, chronic obstructive pulmonary disease obesity, sepsis, liver cirrhosis, or a combination thereof.

19. The method of claim 10, wherein said metabolites of the amino acids and proteins comprises to citrulline, arginine, tyrosine, KIC, leucine, HMB, or a combination thereof.

20. A kit for identifying a disease of an individual based on protein or amino acid metabolic kinetics comprising:
a mixture of isotope labeled amino acids;
instructions for using said mixture for phenotyping amino acids or protein metabolic kinetics via the method of claim 1; and
reference phenotypes comprising an amino acids and/or protein metabolic phenotype from healthy individuals, and a set of amino acids and/or protein metabolic phenotypes from individuals with each of the diseases to be tested.

21. The kit of claim 20, wherein said diseases to be tested comprises chronic heart failure, chronic obstructive pulmonary disease, cancer, obesity, sepsis, liver cirrhosis or a combination thereof.

22. A method for identifying chronic heart failure in an individual in need of such, comprising the steps of:
administering a solution containing L[tau-²H₃]methyl-histidine to said individual and to a control subject;
taking a biological sample periodically from said individual and said control subject;
measuring the amount of L-[tau-²H₃]methyl-histidine and the metabolites thereof in the samples of said individual and said control subject; and
calculating whole body appearance rates of methyl-histidine in said individual and control subject based on the amount of L-[tau-²H₃]methyl-histidine and the metabolites thereof in the biological samples, wherein a higher whole body appearance rate of methyl-histidine in said individual compared to the control subject indicates that said individual has chronic heart failure.

23. The method of claim 22, wherein said solution containing L-[tau-²H₃]methyl-histidine is administered in a pulsed pattern.

24. The method of claim 22, wherein said solution containing L-[tau-²H₃]methyl-histidine is administered intravenously.

25. The method of claim 22, wherein said biological sample is blood or plasma.

26. The method of claim 22, wherein said biological samples are taken periodically at a frequency of about every 15 minutes for about 3 hours.

27. The method of claim 22, wherein the amount of L-[tau-²H₃]methyl-histidine and the metabolites thereof are measured using mass spectrometry.

28. The method of claim 22, wherein said whole body rates of appearance are calculated using a compartmental model.
